(19) United States
(12) Patent Application Publication
(10) Pub. No.: US 2026/0256426 A1
(43) Pub. Date: Sep. 3, 2026

(54) INFLAMMATORY RESPONSE INDICATOR

(71) Applicant: THE ROYAL INSTITUTION FOR THE ADVANCEMENT OF LEARNING / MCGILL UNIVERSITY, Montréal (CA)

(72) Inventors: Amir HADID, Montréal (CA); Dennis JENSEN, Montréal (CA); Qianggang DING, Montréal (CA)

(21) Appl. No.: 19/165,092

(22) PCT Filed: Mar. 14, 2024
(86) PCT No.: PCT/CA2024/050307

§ 371 Date: Sep. 12, 2025

Related U.S. Application Data
(60) Provisional application No. 63/490,445, filed on Mar. 15, 2023.

(51) Int. Cl. A61B 5/00; G16H 10/60

(52) U.S. Cl. CPC A61B 5/7275; A61B 5/4842; A61B 5/7221; A61B 5/7246; G16H 10/60

(57) ABSTRACT

A device, system and method for detecting an inflammatory response modification. A plurality of sensor readings are obtained from an individual over a period of time. Over the period of time, at least a subset of the plurality of sensor readings are computed into a plurality of timestamped records. Applying an inflammatory response variability model on the plurality of timestamped records, an indicator of variation is identified for one or more values for at least one inflammatory biomarker for the individual. A deterioration in a medical condition of the individual may be predicted from the indicator when the inflammatory response variability model indicates a valid increase of the one or more values. An improvement may be predicted from the indicator when the inflammatory response variability model indicates a valid decrease of the one or more values.

### TECHNICAL FIELD

The present invention relates to indicating a likelihood of inflammatory response and, more particularly, to indicating a likelihood of inflammatory response from a plurality of sensor readings.

### BACKGROUND

Diminishing the consequences of a disease or illness is a common aim of many research projects. It is commonplace to closely monitor symptoms of a disease and to treat them as early as possible. Unfortunately, it is very difficult to predict outburst of a disease or improvement prior to some modifications of measurable symptoms. The present disclosure addresses the problem and provides at least a partial solution for indicating a likelihood of an inflammatory response variation prior to or in the absence of symptoms.

### SUMMARY

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In some aspects, the techniques described herein relate to a method for detecting an inflammatory response modification in an individual. The method comprises obtaining a plurality of sensor readings from an individual over a period of time; computing over the period of time at least a subset of the plurality of sensor readings into a plurality of timestamped records; and, by applying an inflammatory response variability model on the plurality of timestamped records, identifying an indicator of variation for one or more values for at least one inflammatory biomarker for the individual.

In some aspects, the techniques described herein relate to a method, further including indicating a likelihood of a deterioration in a medical condition of the individual from the indicator when the inflammatory response variability model indicates a valid increase of the one or more values.

In some aspects, the techniques described herein relate to a method, further including indicating a likelihood of an improvement in a medical condition of the individual from the indicator when the inflammatory response variability model indicates a valid decrease of the one or more values.

In some aspects, the techniques described herein relate to a method wherein the indicator relates to one or more medical conditions. In embodiments, such medical conditions are one or more of an infectious disease, an autoimmune disease, a metabolic syndrome, a sterile inflammation, an exercise induced inflammation, a drug induced inflammation, sarcopenia, a preoperative inflammation, hypertension, a human immunodeficiency virus (HIV) infection (e.g., to monitor inflammation following antiviral therapy), chronic obstructive pulmonary disease (COPD), allergy, asthma, arteriosclerosis and Alzheimer's disease.

In some aspects, the techniques described herein relate to a method wherein obtaining the plurality of sensor readings is performed during a free-living baseline period of the individual, the method further including establishing a baseline pattern for the individual from the plurality of timestamped records obtained there during.

In some aspects, the techniques described herein relate to a method, wherein establishing the baseline pattern for the individual is repeated after a buffer period of at least 1 day following the identifying of the indicator of variation.

In some aspects, the techniques described herein relate to a method further including selecting the subset of the plurality of sensor readings from a set of sensor values of defined nature expected by the inflammatory response variability model, the set of one or more sensor values of defined nature being determined from a correlation factor with the at least one inflammatory biomarker.

In some aspects, the techniques described herein relate to a method wherein the set of sensor values of defined nature is selected from the list consisting of: {minimal heart rate variability measured during a night (hrv_min)}; {standard deviation of heart rate during the night (hr_std)}; and {mean breathing rate during the night (breath_average)}.

In some aspects, the techniques described herein relate to a method wherein the set of sensor values of defined nature is selected from the list consisting of: {BR; HRV}; {BR; HR}; {BR; SLEEP}; {BR; ACTIVITY}; {BR; TEMPERATURE}; {HR; HRV}; {HR; SLEEP}; {HR; BR}; {HR; ACTIVITY}; {HRV; SLEEP}; {HRV; ACTIVITY}; {SLEEP; ACTIVITY}; {TEMPERATURE; HRV}; {TEMPERATURE; SLEEP}; {TEMPERATURE; HR}; {BP; HR}; {BP; HRV}; {BP; BR}; {BP; SLEEP}; {BP; ACTIVITY}; and {BP; TEMPERATURE}.

In some aspects, the techniques described herein relate to a method wherein obtaining the plurality of sensor readings is performed at a frequency between once per night to 265 times per second.

In some aspects, the techniques described herein relate to a method wherein obtaining the plurality of sensor readings is performed from one or more wearable sensing devices worn by the individual. In some aspects, the techniques described herein relate to a method wherein obtaining the plurality of sensor readings is performed from one or more of a ring/watch/shirt/earring/smart contact lens/patch/band/worn by the individual. In some aspects, more than one wearable sensing devices are worn by the individual.

In some aspects, the techniques described herein relate to a method wherein the indicator is identified prior to occurrence of a clinical or clinico-physiological manifestation of a varying inflammatory response by the individual.

### BRIEF DESCRIPTION OF THE DRAWINGS

Further features and exemplary advantages of the present invention will become apparent from the following detailed description, taken in conjunction with the appended drawings, in which:

FIG. 1 is a flow chart of an exemplary method in accordance with the teachings of the present invention;

FIG. 2 is a logical modular representation of an exemplary network node deployed in a system in accordance with the teachings of the present invention;

FIG. 3 is a feature correlation map in accordance with the teachings of the present invention;

FIG. 4 presents visualization graphs of results on two data splits in accordance with the teachings of the present invention;

FIG. 5 presents visualization graphs of receiver operating characteristic (ROC) curves in accordance with the teachings of the present invention;

FIGS. 6 to 13 present visualization graphs of receiver operating characteristic (ROC) curves in accordance with the teachings of the present invention; and

FIGS. 13 to 17 present visualization graphs of predicted normalized inflammatory fold change score curves in accordance with the teachings of the present invention.

### DETAILED DESCRIPTION

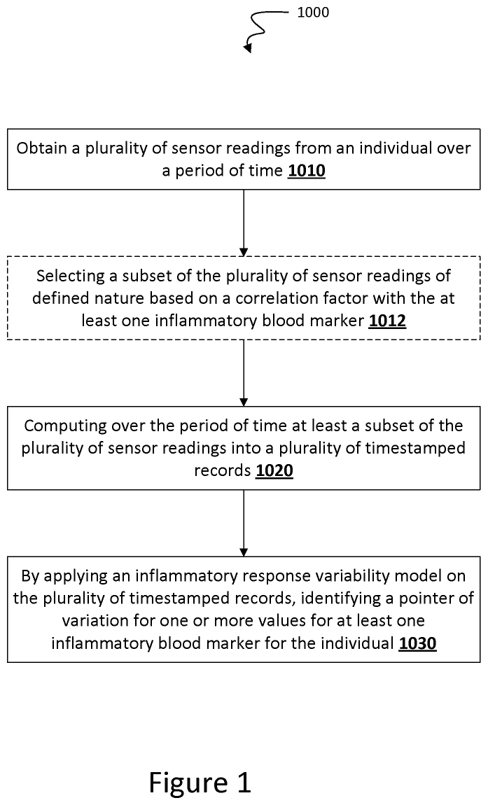

Reference is now made to the drawings in which FIG. 1 shows a flowchart of an example method 1000 for an inflammatory response modification in an individual. In some implementations, one or more process blocks of FIG. 1 may be performed by a device 2100.

As shown in FIG. 1, the method 1000 may include obtaining 1010 a plurality of sensor readings from the individual over a period of time. For example, the device 2100 may obtain a plurality of sensor readings from an individual using one or more wearable sensors or sensor device. Examples of wearable sensors or sensor devices including, without limitation, a ring (e.g., Oura™ ring) a watch or smart watch (e.g., Apple Watch™, a shirt (e.g., Hexoskin™ shirt), one or more earrings, one or more smart contact lens, a patch and a band (e.g., wrist band, ankle ban and/or head band).

As also shown in FIG. 1, the method 1000 may include computing 1020 over the period of time at least a subset of the plurality of sensor readings into a plurality of timestamped records. The method 1000 also comprises, by applying an inflammatory response variability model on the plurality of timestamped records, identifying 1030 an indicator of variation for one or more values for at least one inflammatory biomarker for the individual.

The inflammatory response variability model correlates sensor readings with inflammatory biomarkers, as will be further explained hereinbelow Optionally, the method 1000 may also comprise selecting 1012 the subset of the plurality of sensor readings from a set of sensor values of defined nature expected by the inflammatory response variability model prior to computing 1020. The set of one or more sensor values of defined nature may be determined from a correlation factor with the at least one inflammatory biomarker.

An “inflammatory biomarker” as used herein refers to any marker that is indicative of inflammation, which may be assessed in an individual or a biological sample thereof. In embodiments, such indications of inflammation are based on an increase or a decrease of the level of such a biomarker (e.g., relative to a reference level or baseline, which in embodiments, may be derived from a level associated with the presence of inflammation and/or a level associated with the absence of inflammation). In embodiments, such indications are based on the level or concentration of the biomarker. In embodiments, such a biological sample includes any source from the subject that may contain such a biomarker, including without limitation a bodily fluid, secretion or excretion (e.g., blood, plasma, serum, urine, lymph, pleural effusion, saliva, interstitial fluid, peritoneal fluid, etc.). In an embodiment the biological sample is a blood sample.

In embodiments, such inflammatory biomarkers include one or more of sCD40L, EGF, Eotaxin, FGF-2, FLT-3L, Fractalkine, G-CSF, GM-CSF, GROα, IFN-α2, IFNγ, IL-1α, IL-1β, IL-1RA, IL-2, IL-3, IL-4, IL-5, IL-6, IL-7, IL-8, IL-9, IL-10, IL-12p40, IL-12p70, IL-13, IL-15, IL-17A, IL-17E/IL-25, IL-17F, IL-18, IL-22, IL-27, IP-10, MCP-1, MCP-3, M-CSF, MDC, MIG/CXCL9, MIP-1α, MIP-1β, PDGF-AA, PDGF-AB/BB, RANTES, TGFα, TNFα, TNFβ, VEGF-A, CRP, hsCRP, TRAIL, ESR, Neutrophil count, Lymphocyte count, Eosinophil count, Platelet count, Procalcitonin, CXCL11, Surfactant protein D (SP-D), Club (Clara) cell secretory protein 16 (CC16), Fibrinogen, Amyloid, or a composite score based on these biomarkers.

The method 1000 may include additional implementations, such as any single implementation or any combination of implementations described below and/or in connection with one or more other processes described elsewhere herein.

For instance, the method 1000 may include indicating the likelihood of occurrence of an inflammatory response or inflammation in an asymptomatic individual that is exhibiting subclinical inflammatory response or inflammation. In such a case the method 1000 may be used to predict the likelihood of a future occurrence of an associated medical condition in the individual, and in turn provide an opportunity for prophylactic or therapeutic intervention to, for example prevent, delay the onset of and/or reduce the severity of the medical condition or of one or more symptoms thereof.

In a further embodiment, the method 1000 may include indicating a likelihood of an increase in an inflammatory response or inflammation which in turn may be used to predict a deterioration in a medical condition of the individual from the indicator when the inflammatory response variability model indicates a valid increase of the one or more values. Conversely, the method 1000 may include, alternatively or additionally, indicating a likelihood of a decrease in an inflammatory response or inflammation which in turn may be used to predict an improvement in a medical condition of the individual from the indicator when the inflammatory response variability model indicates a valid decrease of the one or more values. In the context of the examples described herein, validation of an increase or validation of a decrease of the values refers to the prediction being based on a multiple decision trees approach for training and validation of the model (e.g., using a gradient boosting approach such as XGBoost).

Alone or in combination with the previous example, the indicator may relate to one or more medical conditions. In embodiments, such medical conditions are one or more of an infectious disease, an autoimmune disease, a metabolic syndrome, a sterile inflammation, an exercise induced inflammation, a drug induced inflammation, sarcopenia, a preoperative inflammation, hypertension, a human immunodeficiency virus (HIV) infection (e.g., to monitor inflammation following antiviral therapy), chronic obstructive pulmonary disease (COPD), allergy, asthma, arteriosclerosis and Alzheimer's disease.

In some instances, alone or in combination with the examples presented above, obtaining the plurality of sensor readings may be performed during a free-living baseline period of the individual and the method 1000 may then include establishing a baseline pattern for the individual from the plurality of timestamped records obtained there during. Establishing the baseline pattern for the individual may be repeated following the identifying of the indicator of variation. A buffer period (e.g., of at least 1 day) may be necessary between the identifying of the indicator and the repeated establishment of the baseline pattern considering expected recovery time and/or expected stabilization of the inflammatory response. Skilled persons will readily understand that the duration of the buffer period after may be set in accordance with an expected recovery period determined from the condition or illness to which the indicator relates.

As previously noted, the method 1000 may include selecting 1012 the subset of the plurality of sensor readings from a set of sensor values of defined nature expected by the inflammatory response variability model. Through experimentation, it has been determined that the correlation factor between the sensor readings and the at least one inflammatory biomarker varies depending on the nature of the readings (e.g., nature of the parameter of an individual status and/or behavior being measured or read). For instance, it has been determined that a single sensor reading value from the set of sensor values may be a sufficient correlation factor when it is one of a minimal heat rate variability measured during a night (hrv_min), a standard deviation of heart rate during the night (hr_std) and a mean breathing rate during the night (breath_average). Furthermore, it has been determined that two sensor reading values could be a sufficient correlation factor when the tuples are selected from the following list: {BR; HRV}; {BR; HR}; {BR; SLEEP}; {BR; ACTIVITY}; {BR; TEMPERATURE}; {HR; HRV}; {HR; SLEEP}; {HR; BR}; {HR; ACTIVITY}; {HRV; SLEEP}; {HRV; ACTIVITY}; {SLEEP; ACTIVITY}; {TEMPERATURE; HRV}; {TEMPERATURE; SLEEP}; {TEMPERATURE; HR}; {BP; HR}; {BP; HRV}; {BP; BR}; {BP; SLEEP}; {BP; ACTIVITY}; and {BP; TEMPERATURE}. In the preceding list, BR is used for breathing rate signal, HRV is used for heart rate variability signal (variation in the time interval between heartbeats), SLEEP is used as a sleep quality indicator signal, ACTIVITY is used as an activity level indicator signal, TEMPERATURE is used for an individual temperature deviation signal from baseline, BP is used for an individual blood pressure signal (systolic and diastolic where the features are normalized and the Z score is obtained representing the deviation from baseline). Unless specified in the definition of the signal itself, each one of the signals can be min, max, mean or STD (standard deviation), which leads to more than 100 valid combinations.

Heart rate variability (HRV) is a measure of the variation in time between each heartbeat. The variation is controlled by the autonomic nervous system, which is responsible for regulating the body's internal functions, such as breathing, digestion, and heart rate.

HRV can be measured using various methods, such as electrocardiography (ECG), photoplethysmography (PPG), or pulse oximetry. These methods record the electrical activity of the heart or changes in blood flow to determine the time between each heartbeat (interbeat intervals). Then these sets of interbeat intervals are used to calculate the variability using calculations in the time domain (for example, the root mean square of successive differences between normal heartbeats (RMSSD)) or in the frequency domain (for example low frequency and high frequency contents).

Establishing a baseline for HRV involves collecting data over a period of time to determine an individual's typical HRV patterns. The collection of data can be done through repeated HRV measurements over a few days or even weeks, allowing for identification of any trends or changes over time. This baseline can then be used as a reference point for future HRV measurements, providing insight into an individual's overall health and well-being.

A higher HRV is generally associated with better health outcomes, indicating a more flexible and responsive autonomic nervous system. On the other hand, a lower HRV may be indicative of stress, illness, or other factors that may negatively impact overall health.

SLEEP—sleep quality indicator may, for instance, be sleep duration (sum of all sleep stages), bedtime duration (time from bedtime start to bedtime end) or sleep stage duration (time spent in each of the sleep stages such as rapid eye movement (REM), light or deep sleep).

ACTIVITY—activity level indicator can be the total time spent during a day in rest condition, low intensity activity, medium intensity activity, or high intensity activity, calories burned in physical activity, total calories burned, and steps per day.

TEMPERATURE—individual temperature deviation from baseline may be measured frequently (e.g., every one minute) directly from the skin, during the night, and then averaged per night. Then, an average of several nights can be established to create a baseline.

Temperature deviation represents how much the body temperature from an individual's most recent night's sleep changed, either in a positive or negative direction, from this long-term average baseline (e.g., it's +0.3° or −0.2° today compared to individual's normal body temperature).

It has been demonstrated that obtaining the plurality of sensor readings may be performed at a frequency between once per night to 265 times per second. As mentioned below, providing a value from the readings that has been treated to represent one night may be sufficient to ensure a sufficient correlation factor. No matter what the frequency of the readings may be, the plurality of sensor readings may then be statistically manipulated (e.g., average, mode, standard deviance, etc.) considering required inputs from the model. As skilled persons will readily recognize, obtaining the plurality of sensor readings may be performed from one or a plurality of wearable sensing devices worn by the individual or able to measure parameters for the individual (e.g., infrared or visible spectrum camera, remote sensor, etc.).

Experimentation has also demonstrated that the indicator may be identified prior to occurrence of a clinical or clinico-physiological manifestation of a varying inflammatory response by the individual.

Although FIG. 1 shows example blocks of the method 1000, in some implementations, the method 1000 may include additional blocks, fewer blocks, different blocks, or differently arranged blocks than those depicted in FIG. 1. Additionally, or alternatively, two or more of the blocks of the method 1000 may be performed in parallel.

The method 1000 may include, prior to obtaining 1010 the plurality of sensor readings, building (not shown) the inflammatory response variability model during a setup period with a sufficient large group of individuals (referred to herein as subjects for clarity purposes). In such a building phase, a first individualize set of measurements are taken during a first free-living baseline period of the subjects. A second set of individualized measurements is taken during a second free-living response period of the subjects. The first period provides a baseline of inflammatory biomarker values and baseline sensors readings for the subjects. Blood or, more generally, fluid samples may be taken on a daily basis or on a frequency that is determined to be sufficient in the context of the model building phase. Likewise, sensor readings may be taken as previously described in relation to the obtaining 1010 or at another frequency determined to be sufficient in the context of the model building phase. The second period follows exposure of the subjects to an agent or stimulus capable of inducing inflammation and provide a set of response inflammatory biomarker values and response sensors readings for the individual. Again, blood or fluid samples may be taken on a daily basis or on a frequency that is determined to be sufficient in the context of the model building phase, which may be different than the frequency of the first period. In the same manner, sensor readings may be taken as previously described in relation to the obtaining 1010 or at another frequency determined to be sufficient in the context of the model building phase, which may be different from the frequency used in the first period. As such, the baseline inflammatory biomarker values as well as the set of response inflammatory biomarker values are obtained from a plurality of samples from the subjects taken respectively during the first period and the second period. Likewise, the baseline sensors readings and the response sensors readings are obtained from a plurality of sensors readings from the subjects taken respectively during the first period and the second period.

An “agent or stimulus capable of inducing inflammation” refers to any kind of agent or activity/treatment that is known for inducing an inflammatory response or inflammation in a subject, either directly or indirectly. Examples of such agents include for example pathogens or derivatives thereof, lipopolysaccharides and drugs. Examples of such pathogens or derivatives thereof include for example viruses (fully potent, live-attenuated, or inactivated), viroids, prions, bacteria, parasites, fungi and spores. In embodiments such pathogens may be fully potent, live-attenuated, or inactivated, and include for example pathogens or derivatives thereof used in immunization and vaccine compositions. For example, in the case of a respiratory inflammatory condition, such an agent may include a vaccine for a respiratory condition, such as influenza. Examples of such stimuli or activities/treatments include exercise (e.g., high intensity), sleep deprivation, dietary patterns (e.g., a diet high in processed foods, sugar, and unhealthy fats), stress (e.g., public speaking, mental arithmetic, or social rejection). Examples of respiratory diseases include COVID-19, Influenza, RSV, Rhinivirus (common cold), and COPD.

Although FIG. 1 shows example blocks of the method 1000, in some implementations, the method 1000 may include additional blocks, fewer blocks, different blocks, or differently arranged blocks than those depicted in FIG. 1. Additionally, or alternatively, two or more of the blocks of method 1000 may be performed in parallel.

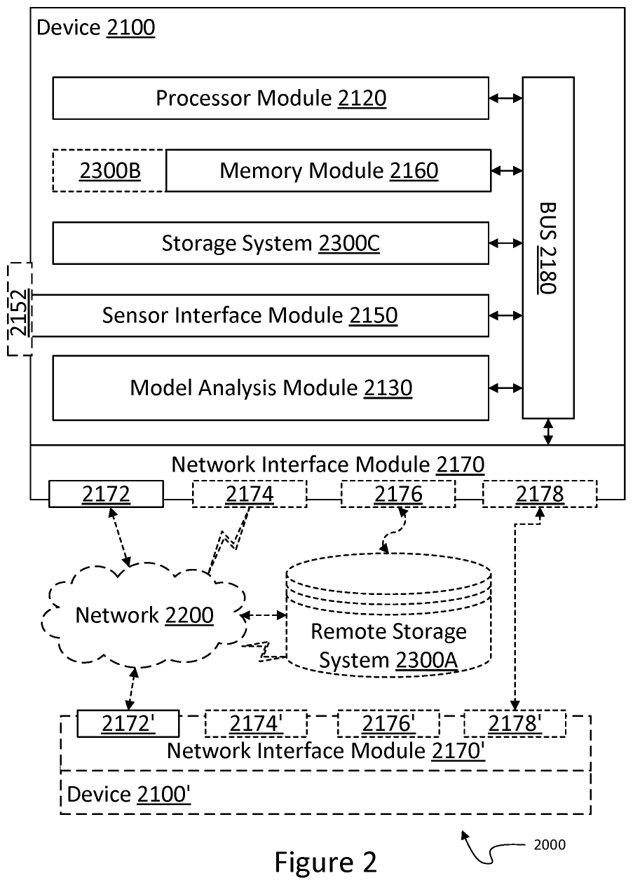

FIG. 2 shows a logical modular representation of an exemplary system 2000 comprising a device 2100. The device 2100 comprises a memory module 2160, a processor module 2120, a model analysis module 2130 and a network interface module 2170. The device 2100 may also include a sensor interface module 2150.

The system 2000 may comprise a storage system 2300 for storing and accessing long-term (i.e., non-transitory) data and may further log data while the device 2100 is being used. FIG. 1 shows examples of the storage system 2300 as a distinct database system 2300A, a distinct module 2300C of the device 2100 or a sub-module 2300B of the memory module 2160 of the device 2100. The storage system 2300 may be distributed over different systems A, B, C. The storage system 2300 may comprise one or more logical or physical as well as local or remote hard disk drive (HDD) (or an array thereof). The storage system 2300 may further comprise a local or remote database made accessible to the device 2100 by a standardized or proprietary interface or via the network interface module 2170.

The network interface module 2170 represents at least one physical interface that can be used to communicate with other network nodes. The network interface module 2170 may be made visible to the other modules of the device 2100 through one or more logical interfaces. The actual stacks of protocols used by the physical network interface(s) and/or logical network interface(s) 2172-2178 of the network interface module 2170 do not affect the teachings of the present invention.

The processor module 2120 may represent a single processor with one or more processor cores or an array of processors, each comprising one or more processor cores. The memory module 2160 may comprise various types of memory (different standardized or kinds of Random Access Memory (RAM) modules, memory cards, Read-Only Memory (ROM) modules, programmable ROM, etc.).

A bus 2180 is depicted as an example of means for exchanging data between the different modules of the device 2100. The teachings presented herein are not affected by the way the different modules exchange information. For instance, the memory module 2160 and the processor module 2120 could be connected by a parallel bus, but could also be connected by a serial connection or involve an intermediate module (not shown) without affecting the teachings of the present invention.

A model analysis module 2130 provides model-computation-related services to the device 2100.

The variants of processor module 2120, memory module 2160 and network interface module 2170 usable in the context of the present invention will be readily apparent to persons skilled in the art. Likewise, even though explicit mentions of the Model analysis module 2130, the memory module 2160, the sensor interface module 2150 and/or the processor module 2120 are not made throughout the description of the present examples, persons skilled in the art will readily recognize when such modules are used in conjunction with other modules of the device 2100 to perform routine as well as innovative elements presented herein.

The following provides an example of context on which the teachings presented herein may be exploited. For instance, based on the results from experimentation, it is anticipated that the teachings may be used for a very sensitive (~90%) and specific (~80%) detection of respiratory disease (influenza and SARS-CoV-2) using data collected from wearable sensors (e.g., ring) and based on objective inflammatory host response quantification, independent of symptoms presence, onset or severity. Skilled persons will readily understand that the example of the ring being used as the sensor is for illustrative purposes and that other sensors (e.g., watch and/or shirt) may also be used alone or in conjunction therewith. A cohort of 55 participants have been formed and a profile was created for each individual in a database. Influenza was used as the agent or stimulus capable of inducing inflammation and was administered via FLUMIST. Data has been gathered from both wearable sensors (e.g., Oura™ ring, Biobeat™ watch, and Astroskin™/Hexoskin™ shirts,) and inflammatory status from blood samples analyses. In the cohort, 4 participants naturally contracted SARS-CoV-2 following/in parallel to the Influenza inoculation. Therefore, there are two kinds of data: 1) Physiological, activity and sleep parameters acquired from the wearable sensors, and 2) Inflammatory status derived from biomarkers assay.

The physiological, activity and sleep data can be described as follows. From the wearable sensors (an Oura™ ring in this example), heart rate (hr) and heart rate variability (hrv) data was gathered at 5-minute intervals collected during sleep periods, and the other parameters have been consolidated data for each night. From the sleep consolidated data available for each night, 9 features were used. In addition, 9 features are added to describe daily activity patterns. The full list of features used for the model training is shown in Table A.

TABLE A
Feature description of consolidated data for each night.

| Feature | Description |
|---|---|
| breath_average | Average respiratory rate. |
| Hr_average | The average heart rate registered during the sleep period. |
| Rmssd | The average HRV calculated with rMSSD method. |
| Temperature_deviation | Skin temperature deviation from the long-term temperature averag |
| temperature_trend_deviation | Skin temperature deviation from weighted three-day rolling average |
| bedtime_start_delta | Number of seconds before or after midnight that bedtime start was |
| | registered |
| bedtime_end_delta | Number of seconds before or after midnight that bedtime end was |
| | registered |
| duration | Total duration of the sleep period (bedtime duration) |
| Total | Total amount of sleep registered during the sleep period |
| cal_active | Energy consumption caused by the physical activity of the day in |
| | kilocalories |
| cal_total | Total energy consumption during the day including Basal Metabolic Rate |
| | in kilocalories |
| daily_movement | Daily physical activity as equal meters i.e., amount of walking needed to |
| | get the same amount of activity |
| steps | Total number of steps registered during the day |
| rest | Number of minutes during the day spent resting i.e., sleeping or lying |
| | down (average MET level of the minute is below 1.05) |
| inactive | Number of inactive minutes (sitting or standing still, average MET level |
| | of the minute between 1.05 and 2) during the day |
| low | Number of minutes during the day with low intensity activity (e.g., |
| | household work) |
| Medium | Number of minutes during the day with medium intensity activity (e.g., |
| | walking) |
| high | Number of minutes during the day with high intensity activity (e.g., |
| | running) |

Inflammatory status from biomarkers assay can be described as follows. For inflammatory status evaluation, 48-plex inflammatory biomarker panel has been used. For each participant, blood was sampled 10 times during the follow-up period: twice prior to inoculation (baseline) and twice daily post inoculation. A score was then computed providing a value for the level of inflammatory host response following exposure to viral respiratory tract infections (VRTI) such as influenza and SARS-CoV-2. The VRTI score is composed of the ratio between the mean values the following 8 biomarkers post vs pre-exposure to the pathogen: IFNγ, IL-6, IL-8, IL-10, IL-15, IP-10, MCP-1, and TNFα (‘fold_change’). The score can provide an estimate of inflammation severity, that can potentially discern between mild and severe VRTI. In addition, when using a threshold for the VRTI score, the score may also indicate when there is an ongoing infection-induced inflammation or not (binary classification). Therefore, the values of pass_threshold were treated as the objective of the model, which is denoted by a binary value 0/1. Therefore, in general, the model answers to a binary classification task. For each participant, the fold_change coefficient of variance (CoV) was computed using 4 values obtained from the baseline period (2 visits, 2 analyses were performed per visit (duplicate)). The computed fold_change value was then used to assist with identifying what is meaningful perturbation to the fold_change, in order to estimate the intra assay, and intra-individual normal variability.

Data preprocessing has been performed as follows:
1. Calculating the minimum, the average, and the standard deviation of the values of ‘hr’ and ‘hrv’, which are represented by ‘hr_min’, ‘hr_mean’, ‘hr_std’, ‘hrv_min’, ‘hrv_mean’, and ‘hrv_std’.
2. Calculating new ‘normalized_fold_change’ values, which will be taken as the objective of the regression model instead of the original ‘fold_change.’ Formally, it can be derived as follows:

$$3. normalized\_fold \_change = fold\_change - average\_cov - 1 \qquad ( 1 )$$

In this way, the normalized_fold_change is consistent alongside all of participants, which means we can simply obtain the pass_threshold by
pass_threshold=sign (normalized_fold_change) for all participants.
4. Filtering out the records with NULL cells or the DateTime is outside the period {−6~4^{th} night}.
5. Normalizing the physiological, activity and sleep data using Z-score method alongside each column independently except temperature_delta, temperature_deviation, and temperature_trend_deviation. The Z-score calculation is as follows:

$${\tilde{f}}_{i} = \frac{{f}_{i} - {f}_{mean}}{{f}_{std}} , where {f}_{mean} = \frac{1}{7} {{\sum }_{j = - 6}}^{0} {f}_{j} and {f}_{std} = \sqrt{\frac{{{\sum }_{j -- 6}}^{0} {( {f}_{j} - {f}_{mean} )}^{2}}{7}}$$

where f denotes an arbitrary kind of feature.
6. Randomly splitting the participants into three folds (train/validation/test sets) with the ratio 35:10:10.

After all of the procedures of data processing, a matrix M was obtained in shape (N*T)×F, where N=55 denotes the number of participants, T=11 denotes the number of days (nights) in the period −6 to 4 days relative to inoculation day, and F=23 denotes the number of features. Among them, 35*T rows were randomly selected as the training set, 10*T rows as the validation set, and another 10*T rows as the testing set.

In terms of methodology, the pipeline may be defined as solving a binary classification problem in two steps:
1. Firstly, the data is acquired from the wearable to detect and/or track the level of inflammation (fold_change). A machine learning-based model is developed for that purpose, which takes M as the input and outputs a value predicted_fold_change to approximate the normalized_fold_change.
2. Then, a threshold is set to predicted_fold_change for quantification thereof to either 0 or 1 (host response inflammation ‘not detected’ or ‘detected’, respectively).

In terms of model, a gradient-boosted decision tree machine learning model as been used as a base model for regression. Specifically, the model takes the training set Mtrain∈(Ntrain*T)×F as the input and outputs Otrain∈R^{Ntrain*T} representing the predicted fold changes. To improve the generalization of the model, a 5-fold cross-validation method has been used to tune the hyper-parameters of the model. The final list of hyper-parameters selected is shown in Table B.

TABLE B
The list of hyper-parameters in XGBoost.

| Name | Explored Range | Value | Explanation |
|---|---|---|---|
| Booster | [‘gbtree’, ‘gblinear’] | gbtree | Type of booster in XGBoost. |
| Learning_rate | [0.01, 0.05, 0.1, 0.5] | 0.05 | Learning rate. |
| N_estimators | [50, 100, 150] | 100 | Number of estimators in XGBoost. |
| Max_depth | [1, 6, 11, 16] | 11 | Maximum depth of a tree. |
| Min_child_weight | [1, 6, 11, 16] | 1 | Minimum sum of instance weight (hessian) needed in a child. |
| Subsample | [0.1, 0.3, 0.5, 0.7, 0.9] | 0.9 | Subsample ratio of the training instances. |
| Colsample_bytree | [0.1, 0.3, 0.5, 0.7, 0.9] | 0.7 | Subsample ratio of columns when constructing each tree. |
| Gamma | Manually set | 0 | Minimum loss reduction. |
| Reg_alpha | Manually set | 10 | L1 regularization term on weights. |
| Reg_lambda | Manually set | 10 | L2 regularization term on weights. |

At the quantification step, M was provided to the trained model leading to the output Oₜᵣₐᵢₙ∈R_{ntrain*T}. After that, a threshold t can be set to quantify O to a vector of binary codes P∈{0, 1}_{N*T}, which can be formulated as follows:

$$P - I ( O > t ) \qquad ( 3 )$$

where P is the final prediction for this binary classification task.

In the example of experiment, the two schemes of data split were considered, as follows:
1. Covid-Aware: The participants tested positive for SARS-CoV-2 are remained in the training set.
2. Covid-Ignore: The participants tested positive for SARS-CoV-2 are removed from the training set.

Three metrics schemes were designed to measure the performance of the model as follows:
1. Night-to-Night: The predicted classes should be exactly consistent with the true labels (detected vs not detected) night-to-night.
2. 1-Tolerated: In this scheme, an actual ‘detected’ inflammatory response that occurred within 24 hours window from the predicted ‘detected’ response was considered to be the same event. Therefore, a prediction is considered as ‘detected’ if any one of the true labels of tonight (day d), last night (day d−1), and tomorrow night (day d+1) is positive, which means we set a tolerance of 1 night to Night-to-Night.
3. Aggregated (optional): All night results were aggregated for each participant. Specifically, a specific participant was considered as positive if the true label of any of the 10 nights is positive. The same does for the prediction. In this way, the number of samples is decreased from N*T to N.

The metric used is ROC-AUC. The Receiver Operator Characteristic (ROC) curve is a probability curve that plots the TPR (True Positive Rate) against FPR (False Positive Rate) at various threshold values. The Area Under the Curve (AUC) is the measure of the ability of a classifier.

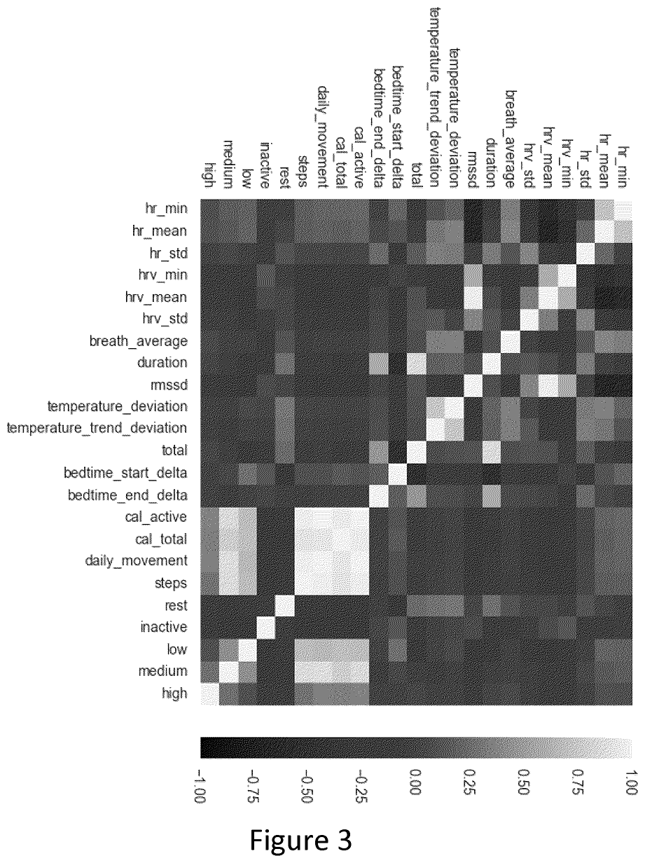

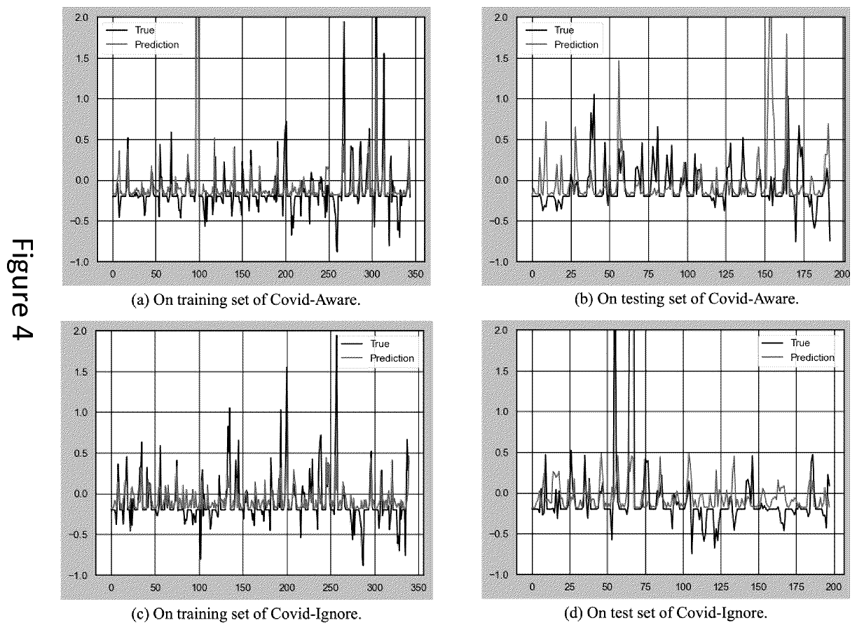

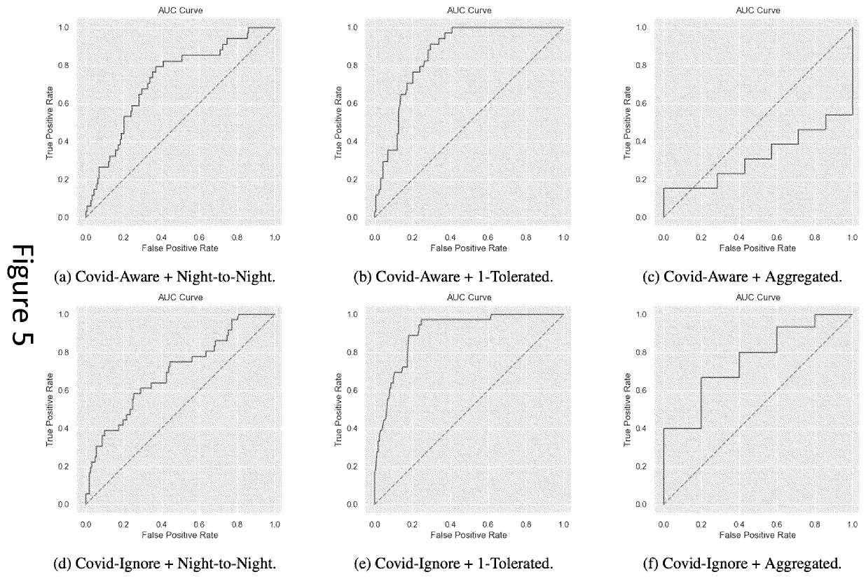

Feature importance and feature correlation map are shown in Table C and FIG. 3, respectively. The visualization of the training and test sets results is shown in FIG. 4. On FIG. 4, black lines denote the actual normalized fold change (change in inflammatory VRTI score) for all timepoints for all the participants. Gray lines denote predicted normalized fold change for the same timepoints. Since a cross-validation method as been used for hyper-parameter tuning, the validation set and the test set are merged in a final test set. The visualization of receiver operating characteristic (ROC) curves is shown in FIG. 5 and the experimental results of ROC-AUC are summarized in Table D.

TABLE C
Feature importance

| COVID-IGNORE | | COVID-AWARE | |
|---|---|---|---|
| hr_min | 0.232967 | temperature_deviation | 0.312765 |
| total | 0.154980 | hr_mean | 0.271300 |
| duration | 0.124490 | rest | 0.149720 |
| cal_active | 0.065024 | hr_min | 0.065335 |
| breath_average | 0.057815 | breath_average | 0.056027 |
| rest | 0.050267 | temperature_trend_deviation | 0.030321 |
| steps | 0.039682 | duration | 0.018641 |
| hr_mean | 0.032743 | hr_std | 0.018169 |
| medium | 0.030224 | hrv_min | 0.017352 |
| hrv_min | 0.027821 | hrv_mean | 0.012688 |
| daily_movement | 0.027269 | high | 0.012586 |
| bedtime_start_delta | 0.026291 | bedtime_end_delta | 0.007164 |
| hrv_mean | 0.024463 | hrv_std | 0.005681 |
| cal_total | 0.022990 | total | 0.005137 |
| low | 0.015422 | medium | 0.003753 |
| high | 0.015239 | inactive | 0.002674 |
| bedtime_end_delta | 0.013645 | rmssd | 0.002281 |
| temperature_trend_deviation | 0.013603 | bedtime_start_delta | 0.002219 |
| hrv_std | 0.011091 | steps | 0.001961 |
| rmssd | 0.007307 | cal_total | 0.001895 |
| hr_std | 0.003568 | daily_movement | 0.001432 |
| temperature_deviation | 0.001670 | low | 0.000842 |
| inactive | 0.001429 | cal_active | 0.000057 |

TABLE D
The experimental results summarized
using ROC area under the curve (AUC)

Scheme

| Data split | Night-to-Night | 1-Tolerated | Aggregated |
|---|---|---|---|
| Covid-Aware | 0.721 | 0.857 | 0.318 |
| Covid-Ignore | 0.696 | 0.904 | 0.760 |

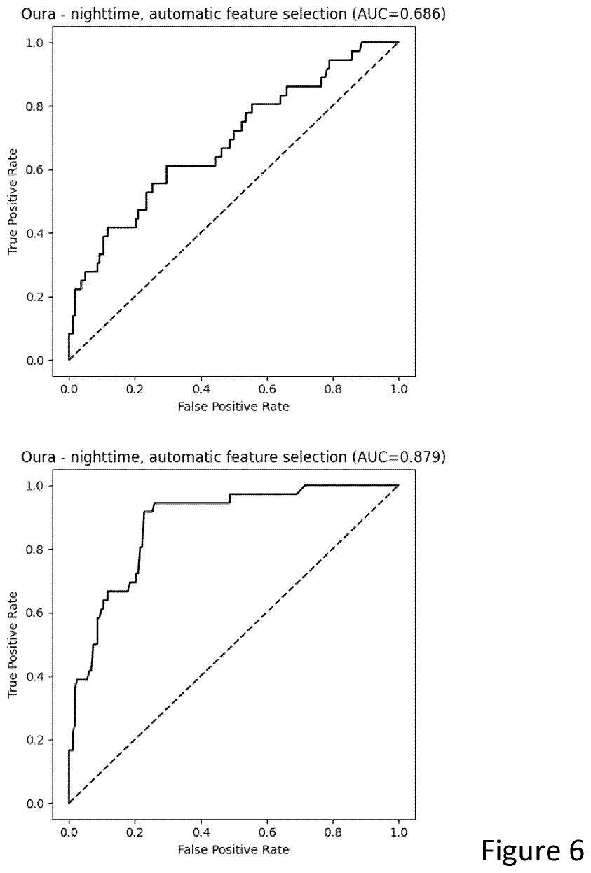

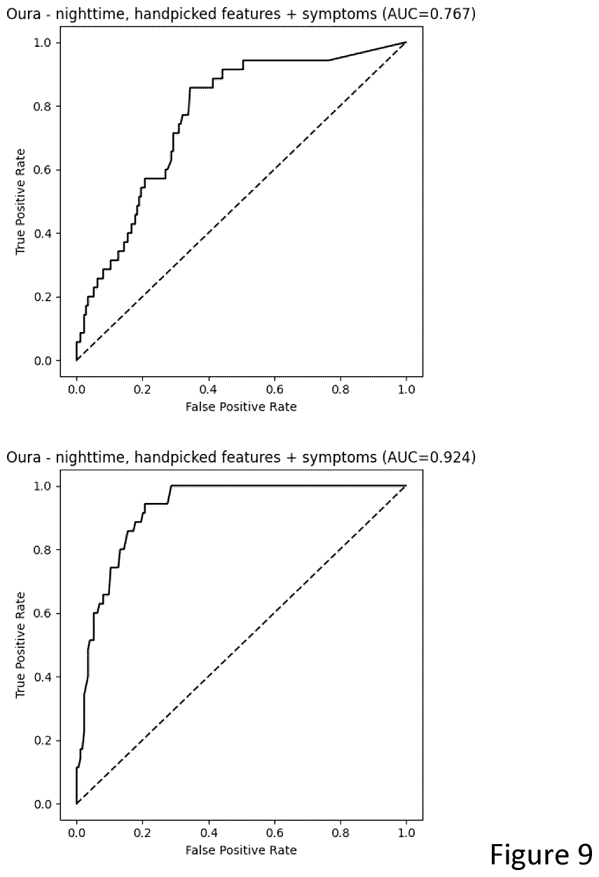

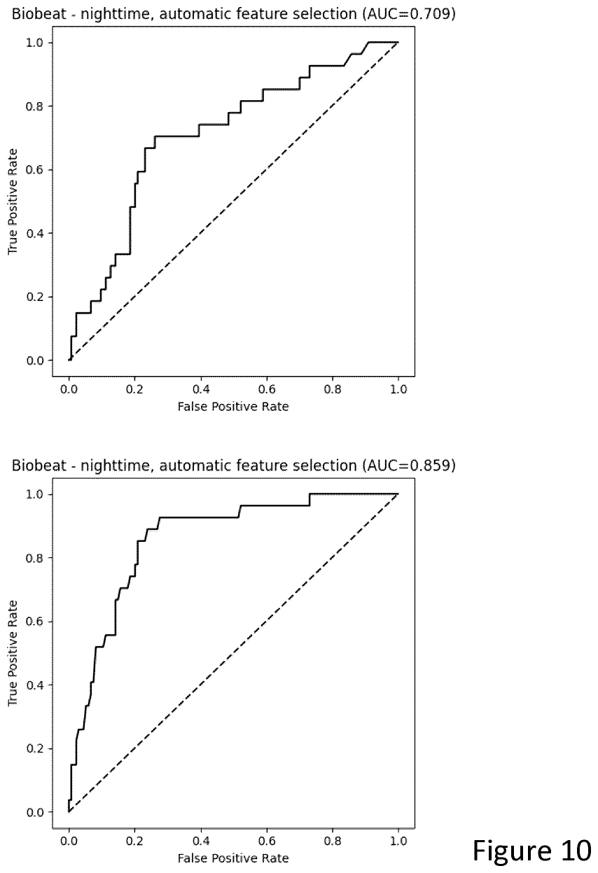

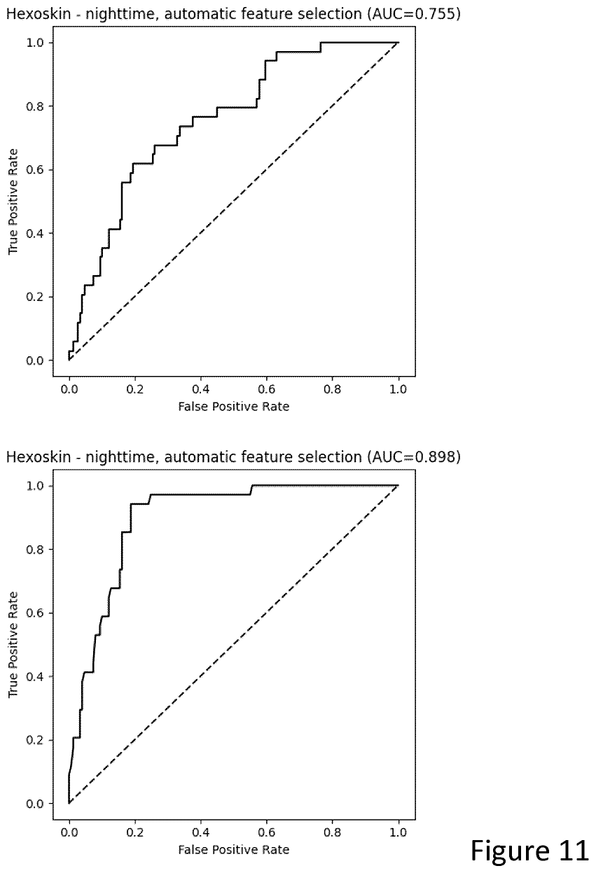

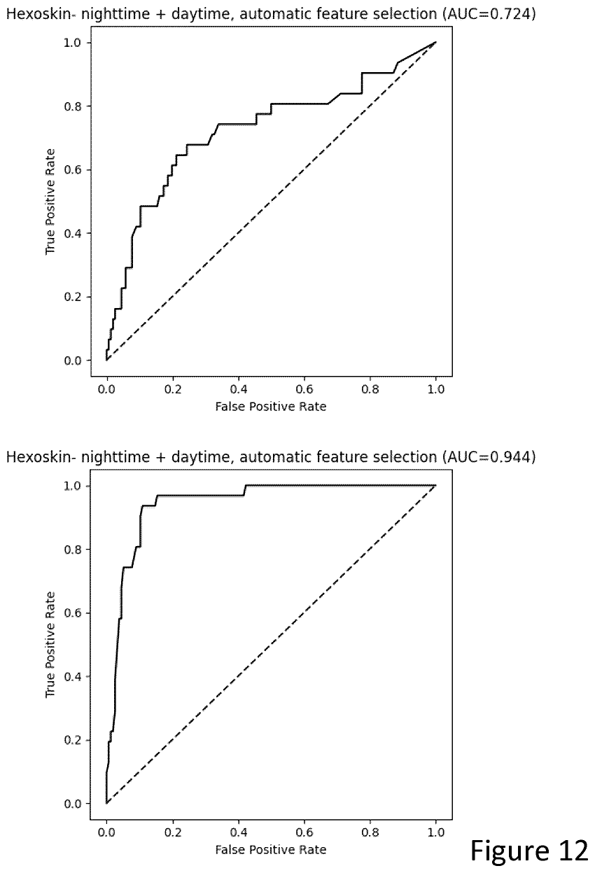

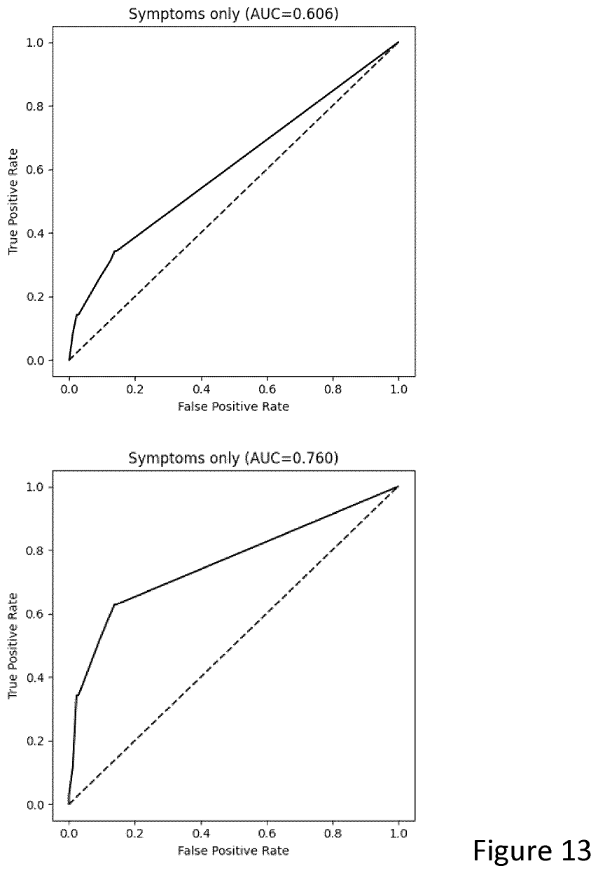

FIGS. 6 to 13 depict performance graphs of selected prediction models (AUC-ROC) using data acquired using a ring (FIGS. 6-9), smart watch (FIG. 10), smart shirt (FIGS. 11-12) and symptoms only as a reference model (FIG. 13). Each Figure related to a model evaluated using the following two schemes: Night to night (upper panel): Realtime comparison of predicted detected inflammatory status compared with true label inflammatory status. 1-Tolerated (lower panel): Detection of inflammatory status with 24-hour tolerance between predicted and actual event.

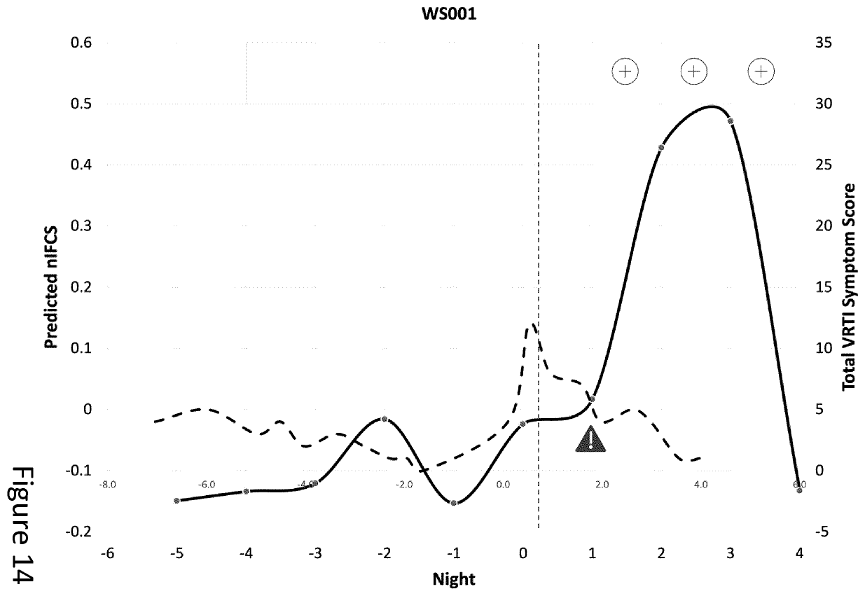

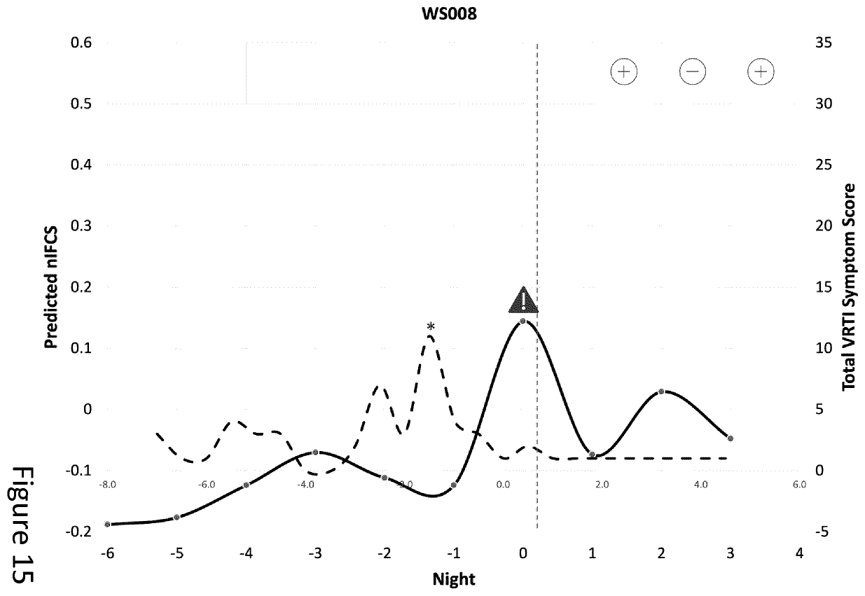

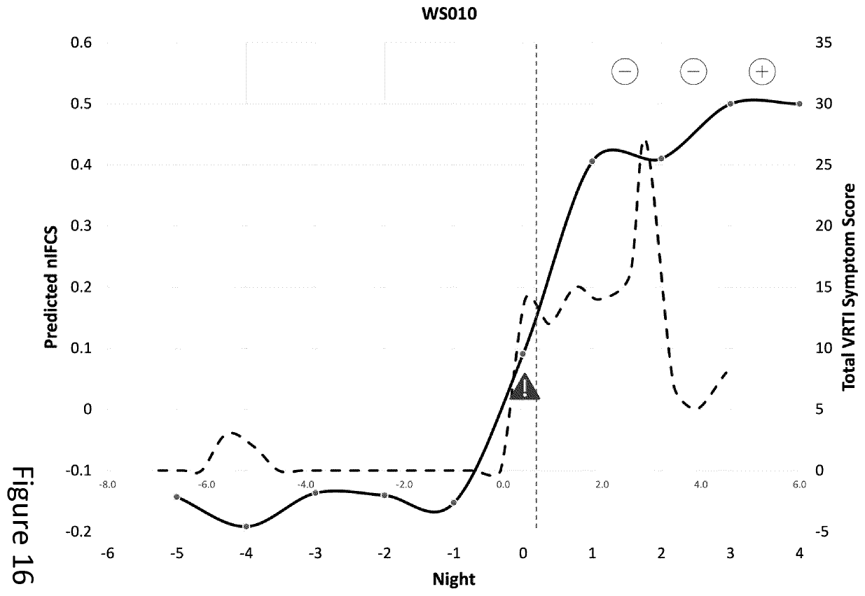

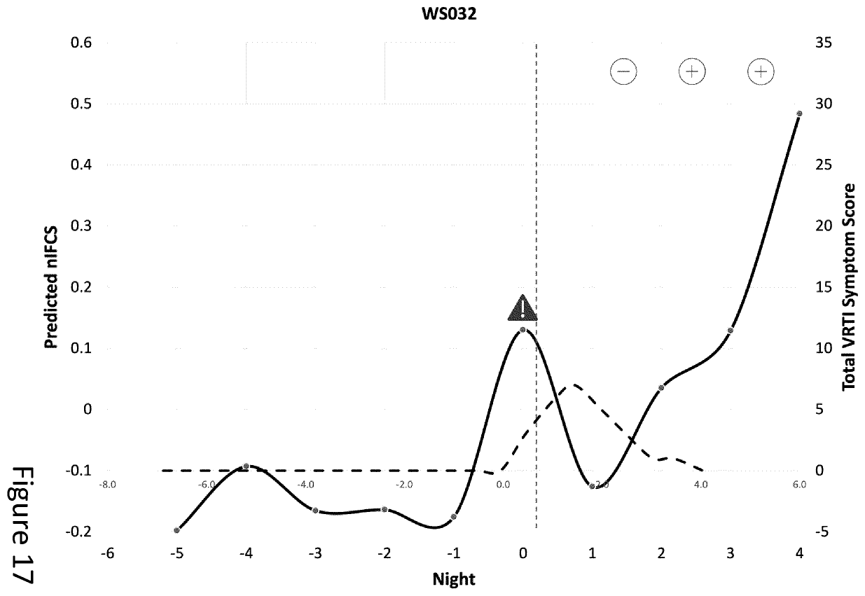

FIGS. 14 to 17 depict model performance graphs showing predicted normalized inflammatory fold change score (nIFCS; left y-axis) and total viral respiratory tract infection (VRTI) symptom score (right y-axis) of four participants excluded from training/validation sets because they tested positive by PCR for SARS-CoV-2 infection following inoculation with the live attenuated influenza vaccine, which is marked by the dashed vertical line. Detection as “inflamed” by the prediction model is marked by a grey alert icon. Positive SARS-CoV-2 test is marked by ⊕. Negative SARS-CoV-2 test is marked by ⊖. * denotes symptoms not related to respiratory illness (menstrual cycle).

TABLE E
Feature importance list for the example depicted on
FIG. 6 (Oura, nighttime, auto feature selection).

| Feature | Importance |
|---|---|
| Oura hr_min | 0.21338198 |
| Oura hrv_max | 0.13718775 |
| Oura total | 0.10255181 |
| Oura breath_average | 0.07400942 |
| Oura duration | 0.05224343 |
| Oura rest | 0.04200281 |
| Oura cal_active | 0.03777463 |
| Oura cal_total | 0.02749465 |
| Oura temperature_delta | 0.02713488 |
| Oura hr_average | 0.02669654 |
| Oura hrv_std | 0.02509922 |
| Oura bedtime_start_delta | 0.02297067 |
| Oura temperature_trend_deviation | 0.0213034 |
| Oura daily_movement | 0.0200247 |
| Oura hrv_mean | 0.01883794 |
| Oura low | 0.01848711 |
| Oura hr_mean | 0.01826131 |
| Oura hrv_p1 | 0.01798985 |
| Oura hr_max | 0.0177895 |
| Oura medium | 0.0147549 |
| Oura hr_std | 0.01336091 |
| Oura bedtime_end_delta | 0.01322796 |
| Oura high | 0.01093469 |
| Oura steps | 0.01051151 |
| Oura hr_p1 | 0.01004044 |
| Oura hrv_min | 0.00512021 |
| Oura inactive | 0.00080776 |

Legend:
hr = heart rate, hrv = heart rate variability, total = total sleep time, breath_average = average respiration rate, duration = time in bed, cal_active = energy consumption caused by the physical activity of the day, cal_total = total energy consumption during the day including Basal Metabolic Rate, std = standard deviation, bedtime_start_delta = number of seconds before or after midnight that bedtime start was registered, temperature_trend_deviation = skin temperature deviation from weighted three-day rolling average, daily_movement = daily physical activity as equal meters, low = number of minutes during the day with low intensity activity, p1 = slope of the linear proximation, max = maximal value, temperature_delta = 7kin temperature deviation from the long-term temperature average, bedtime_end_delta = number of seconds before or after midnight that bedtime end was registered, high = number of minutes during the day with high intensity activity, steps = total number of steps registered during the day, min = minimal value, inactive = number of inactive minutes.

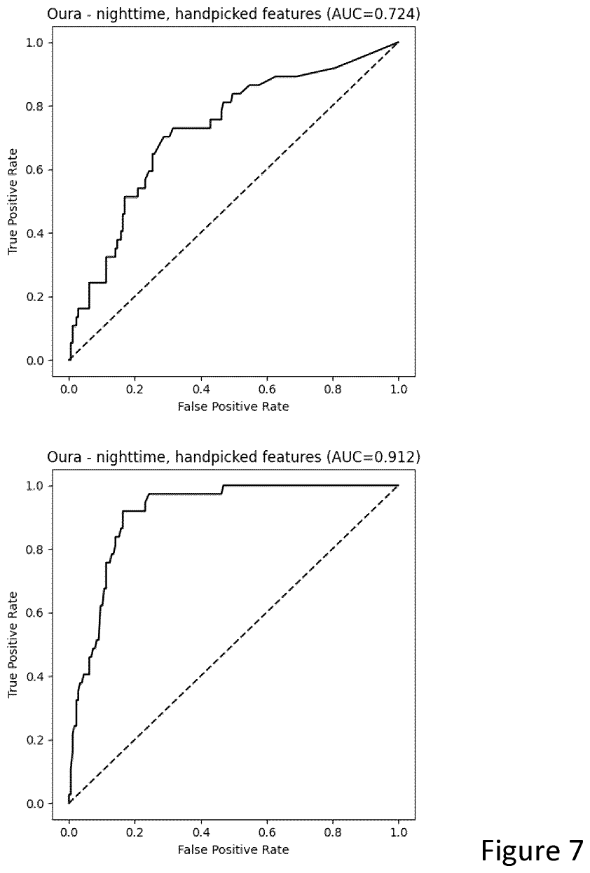

TABLE F
Feature importance list for the example depicted
on FIG. 7 (Oura, nighttime, handpicked features).

| Feature | Importance |
|---|---|
| hrv_max | 0.27839997 |
| hr_min | 0.25228915 |
| breath_average | 0.13884702 |
| hr_mean | 0.06634278 |
| hrv_mean | 0.048537254 |
| cal_total | 0.04755316 |
| hrv_p1 | 0.04634978 |
| temperature_deviation | 0.037400316 |
| hr_p1 | 0.03279607 |
| hr_max | 0.03192166 |
| hrv_min | 0.019562807 |

Legend:
hrv = heart rate variability hr = heart rate, breath_average = average respiration rate, cal_total = total energy consumption during the day including Basal Metabolic Rate, temperature_deviation = skin temperature deviation from the long-term temperature average, min = minimal value, max = maximal value, p1 = slope of the linear proximation.

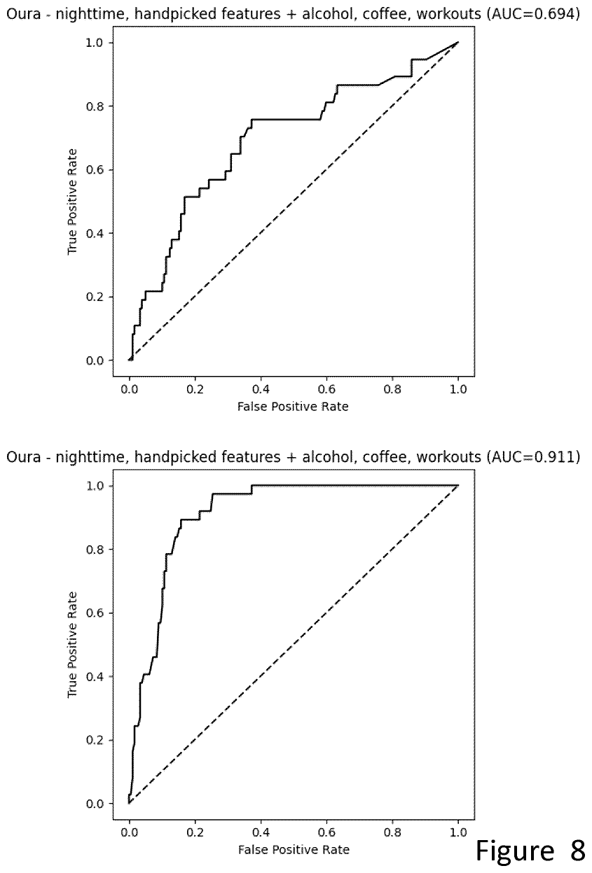

TABLE G
Feature importance list for the example depicted on FIG. 8 (Oura,
nighttime, handpicked features + alcohol, coffee, workouts).

| Feature | Importance |
|---|---|
| hrv_max | 0.24429394 |
| hr_min | 0.24128085 |
| Annotation alcohol | 0.11638646 |
| breath_average | 0.102105096 |
| hrv_mean | 0.04693097 |
| hr_mean | 0.043811195 |
| hrv_p1 | 0.042473555 |
| temperature_deviation | 0.034715954 |
| cal_total | 0.03157264 |
| hr_max | 0.025821645 |
| Annotation workout | 0.02404483 |
| hr_p1 | 0.022722987 |
| Annotation caffeine | 0.018109016 |
| hrv_min | 0.005730864 |

hrv = heart rate variability hr = heart rate, alcohol = number of alcoholic drinks consumed before bedtime, breath_average = average respiration rate, temperature_deviation = skin temperature deviation from the long-term temperature average, cal_total = total energy consumption during the day including basal metabolic rate, workout = workout intensity annotated before bedtime, caffeine = number of caffeinated drinks consumed before bedtime, min = minimal value, max = maximal value, p1 = slope of the linear proximation.

TABLE H
Feature importance list for the example depicted on FIG.
9 (Oura, nighttime, handpicked features + symptoms).

| Feature | Importance |
|---|---|
| hrv_max | 0.23830818 |
| hr_min | 0.21718155 |
| symptom | 0.18802904 |
| breath_average | 0.12157255 |
| cal_total | 0.0374661 |
| temperature_deviation | 0.035520792 |
| hr_mean | 0.033572607 |
| hrv_min | 0.033278786 |
| hrv_p1 | 0.030902378 |
| hr_max | 0.02409112 |
| hrv_mean | 0.0212337 |
| hr_p1 | 0.018843181 |

Legend:
hrv = heart rate variability hr = heart rate, symptom = total symptom score, breath_average = average respiration rate, cal_total = total energy consumption during the day including basal metabolic rate, temperature_deviation = skin temperature deviation from the long-term temperature average, min = minimal value, max = maximal value, p1 = slope of the linear proximation.

TABLE I
Feature importance list for the example depiected on
FIG. 10 (Biobeat, nighttime, auto feature selection).

| Feature | Importance |
|---|---|
| rr_std | 0.17698385 |
| hrv_mean | 0.12083445 |
| hr_max | 0.08007494 |
| dbp_max | 0.0749238 |
| rr_min | 0.06738378 |
| sbp_max | 0.06376653 |
| rr_mean | 0.05438074 |
| dbp_std | 0.041037075 |
| hrv_p1 | 0.039532356 |
| dbp_mean | 0.02868999 |
| hrv_max | 0.027243277 |
| sbp_std | 0.022517804 |
| hr_min | 0.021884812 |
| sbp_min | 0.021028452 |
| dbp_min | 0.019309655 |
| rr_p1 | 0.018795466 |
| hr_mean | 0.0187014 |
| dbp_p1 | 0.018508358 |
| hrv_std | 0.016286053 |
| sbp_p1 | 0.01603848 |
| hrv_min | 0.015940916 |
| hr_p1 | 0.014662578 |
| sbp_mean | 0.0109922215 |
| rr_max | 0.010132874 |
| hr_std | 0.0003501984 |

Legend:
rr = average respiration rate, hrv = heart rate variability hr = heart rate, dbp = diastolic blood pressure, sbp = systolic blood pressure, std = standard deviation, min = minimal value, max = maximal value, p1 = slope of the linear proximation.

TABLE J
Feature importance list for the example depicted on FIG.
11 (Hexoskin, nighttime, auto feature selection).

| Feature | Importance |
|---|---|
| hrv_lfnu | 0.2512528 |
| hrv_lf_hf_ratio | 0.0988831 |
| rr_max | 0.09646539 |
| hrv_mean | 0.08079895 |
| hrv_hfnu | 0.06976724 |
| hr_mean | 0.057090558 |
| rr_mean | 0.054486677 |
| rr_p1 | 0.038894713 |
| rmssd_resp | 0.03418511 |
| hrv_max | 0.032614898 |
| hrv_entropy | 0.031132504 |
| rr_min | 0.024085477 |
| mva_mean | 0.01954185 |
| mva_min | 0.018835142 |
| mva_p1 | 0.017342117 |
| mva_std | 0.014898397 |
| hr_p1 | 0.012793986 |
| hrv_std | 0.012659145 |
| mva_max | 0.01151657 |
| hrv_p1 | 0.009335066 |
| hrv_min | 0.005465939 |
| rr_std | 0.0045261527 |
| hr_min | 0.0017600746 |
| hr_max | 0.0012498925 |
| hr_std | 0.00041827533 |

hrv_lfnu = heart rate variability low frequency power in normalized units, hrv_lf_hf_ratio = heart rate variability low frequency to high frequency ratio rr = average respiration rate, hrv = heart rate variability hr = heart rate, rmssd_resp = root mean square of successive differences of the respiration rate, mva = minute ventilation adjusted to body size, std = standard deviation, min = minimal value, max = maximal value, p1—slope of the linear proximation, entropy—entropy of the RR intervals.

TABLE K
Feature importance list for the example depicted on FIG. 12
(Hexoskin, nighttime + daytime, auto feature selection).

| Feature | Importance |
|---|---|
| hrv_hfnu | 0.15784746 |
| mva_max | 0.13925967 |
| mva_mean | 0.09526221 |
| hrv_entropy | 0.0701104 |
| rr_std | 0.067138806 |
| hrv_lf_hf_ratio | 0.064946346 |
| hr_min | 0.056508757 |
| hrv_max | 0.05630612 |
| hrv_lfnu | 0.05325074 |
| mva_p1 | 0.033682507 |
| hrv_std | 0.030588869 |
| mva_min | 0.028972354 |
| rr_mean | 0.022743102 |
| hrv_mean | 0.021644797 |
| rr_min | 0.018468238 |
| hr_mean | 0.016237784 |
| hr_max | 0.013202133 |
| rr_p1 | 0.0129250055 |
| hrv_min | 0.012481162 |
| hr_std | 0.011318607 |
| mva_std | 0.006797943 |
| hrv_p1 | 0.0041037123 |
| mins_sed | 0.0036728878 |
| hr_p1 | 0.0015737031 |
| rr_max | 0.00095666904 |

Legend:
hrv_lfnu = heart rate variability low frequency power in normalized units, mva = minute ventilation adjusted to body size, rr = average respiration rate, hrv_lf_hf_ratio = heart rate variability low frequency to high frequency ratio, hr = heart rate, mins_sed = minutes sedentary, std—standard deviation, min—minimal value, max—maximal value, p1—slope of the linear proximation, entropy—entropy of the RR intervals.

Various network links may be implicitly or explicitly used in the context of the present invention. While a link may be depicted as a wireless link, it could also be embodied as a wired link using a coaxial cable, an optical fiber, a category 5 cable, and the like. A wired or wireless access point (not shown) may be present on the link between. Likewise, any number of routers (not shown) may be present and part of the link, which may further pass through the Internet.

The present invention is not affected by the way the different modules exchange information between them. For instance, the memory module and the processor module could be connected by a parallel bus but could also be connected by a serial connection or involve an intermediate module (not shown) without affecting the teachings of the present invention.

A method is generally conceived to be a self-consistent sequence of steps leading to a desired result. These steps require physical manipulations of physical quantities. Usually, though not necessarily, these quantities take the form of electrical or magnetic/electromagnetic signals capable of being stored, transferred, combined, compared, and otherwise manipulated. It is convenient at times, principally for reasons of common usage, to refer to these signals as bits, values, parameters, items, elements, objects, symbols, characters, terms, numbers, or the like. It should be noted, however, that all of these terms and similar terms are to be associated with the appropriate physical quantities and are merely convenient labels applied to these quantities. The description of the present invention has been presented for purposes of illustration but is not intended to be exhaustive or limited to the disclosed embodiments. Many modifications and variations will be apparent to those of ordinary skill in the art. The embodiments were chosen to explain the principles of the invention and its practical applications and to enable others of ordinary skill in the art to understand the invention in order to implement various embodiments with various modifications as might be suited to other contemplated uses.

1. A method for detecting an inflammatory response modification in an individual, the method comprising:
obtaining a plurality of sensor readings from an individual over a period of time;
computing over the period of time at least a subset of the plurality of sensor readings into a plurality of timestamped records; and
by applying an inflammatory response variability model on the plurality of timestamped records, identifying an indicator of variation for one or more values for at least one inflammatory biomarker for the individual.

2. The method of claim 1, wherein the indicator relates to a medical condition and the method further comprises indicating a likelihood of deterioration in the medical condition in the individual from the indicator when the inflammatory response variability model indicates a valid increase of the one or more values.

3. The method of claim 2, wherein the individual is asymptomatic for the condition when the plurality of sensor readings is obtained, and the method further comprises predicting a likelihood of the future occurrence of the medical condition or one or more symptoms thereof in the individual from the indicator when the inflammatory response variability model indicates a valid increase of the one or more values.

4. The method of claim 1, wherein the indicator relates to a medical condition and the method further comprises indicating a likelihood of an improvement in the medical condition in the individual from the indicator when the inflammatory response variability model indicates a valid decrease of the one or more values.

5. The method of claim 2, wherein the medical condition is one or more of a respiratory disease, an infectious disease, an autoimmune disease, a metabolic syndrome, a sterile inflammation, an exercise induced inflammation, a drug induced inflammation, sarcopenia, a preoperative inflammation, hypertension, a human immunodeficiency virus (HIV) infection, chronic obstructive pulmonary disease (COPD), allergy, asthma, arteriosclerosis and Alzheimer's disease.

6. (canceled)

7. The method of claim 1, wherein obtaining the plurality of sensor readings is performed during a free-living baseline period of the individual, the method further comprising establishing a baseline pattern for the individual from the plurality of timestamped records obtained there during.

8. The method of claim 7, wherein establishing the baseline pattern for the individual is repeated after a buffer period of at least 1 day following the identifying of the indicator of variation.

9. The method of claim 1, further comprising selecting the subset of the plurality of sensor readings from a set of sensor values of defined nature expected by the inflammatory response variability model, the set of one or more sensor values of defined nature being determined from a correlation factor with the at least one inflammatory biomarker.

10. (canceled)

11. The method of claim 9, wherein the set of sensor values of defined nature is selected from the list consisting of:
{BR; HRV}; {BR; HR}; {BR; SLEEP}; {BR; ACTIVITY}; {BR; TEMPERATURE}; {HR; HRV}; {HR; SLEEP}; {HR; BR}; {HR; ACTIVITY}; {HRV; SLEEP}; {HRV; ACTIVITY}; {SLEEP; ACTIVITY}; {TEMPERATURE; HRV}; {TEMPERATURE; SLEEP}; {TEMPERATURE; HR}}; {BP; HR}; {BP; HRV}; {BP; BR}; {BP; SLEEP}; {BP; ACTIVITY}; and {BP; TEMPERATURE}.

12. The method of claim 1, wherein obtaining the plurality of sensor readings is performed at a frequency between once per night to 265 times per second.

13. The method of claim 1, wherein obtaining the plurality of sensor readings is performed from more than one wearable sensing devices worn by the individual.

14. The method of claim 1, wherein the indicator is identified prior to occurrence of a clinical manifestation of a varying inflammatory response by the individual.

15. A system for detecting an inflammatory response modification in an individual comprising:
one or more processors configured to:
obtain a plurality of sensor readings from an individual over a period of time;
compute over the period of time at least a subset of the plurality of sensor readings into a plurality of timestamped records;
by applying an inflammatory response variability model on the plurality of timestamped records, identify an indicator of variation for one or more values for at least one inflammatory biomarker for the individual.

16. The system of claim 15, wherein the indicator relates to a medical condition and the system further comprises indicating a likelihood of a deterioration in the medical condition in the individual from the indicator when the inflammatory response variability model indicates a valid increase of the one or more values.

17. The system of claim 16, wherein the individual is asymptomatic for the condition when the plurality of sensor readings are obtained, and the method further comprises predicting a likelihood of the future occurrence of the medical condition or one or more symptoms thereof in the individual from the indicator when the inflammatory response variability model indicates a valid increase of the one or more values.

18. The system of claim 15, wherein the indicator relates to a medical condition and the system further comprises indicating a likelihood of an improvement in the medical condition in the individual from the indicator when the inflammatory response variability model indicates a valid decrease of the one or more values.

19. (canceled)

20. (canceled)

21. The system of claim 15, wherein obtaining the plurality of sensor readings is performed during a free-living baseline period of the individual, the method further comprising establishing a baseline pattern for the individual from the plurality of timestamped records obtained there during.

22. The system of claim 21, wherein establishing the baseline pattern for the individual is repeated after a buffer period of at least 1 day following the identifying of the indicator of variation.

23. The system of claim 15, further comprising selecting the subset of the plurality of sensor readings from a set of sensor values of defined nature expected by the inflammatory response variability model, the set of one or more sensor values of defined nature being determined from a correlation factor with the at least one inflammatory biomarker.

24. (canceled)

25. The system of claim 15, wherein obtaining the plurality of sensor readings is performed at a frequency between once per night to 265 times per second.

26. The system of claim 15, wherein obtaining the plurality of sensor readings is performed from more than one wearable sensing devices worn by the individual.

27. The system of claim 15, wherein the indicator is identified prior to occurrence of a clinical manifestation of a varying inflammatory response by the individual.

28. (canceled)
